(19) United States
(12) Patent Application Publication
(10) Pub. No.: US 2021/0173032 A1
(43) Pub. Date: Jun. 10, 2021

(54) SHORT T2 TISSUE IMAGING WITH T2 PREP PETRA SEQUENCE

(71) Applicant: Siemens Healthcare GmbH, Erlangen (DE)

(72) Inventors: Fang Dong, Shenzhen (CN); Kun Zhou, Shenzhen (CN)

(73) Assignee: Siemens Healthcare GmbH, Erlangen (DE)

(21) Appl. No.: 17/116,146

(22) Filed: Dec. 9, 2020

(30) Foreign Application Priority Data
Dec. 10, 2019 (CN) 201911258002.X

(51) Int. Cl. G01R 33/561; G01R 33/50; A61B 5/055

(52) U.S. Cl. CPC G01R 33/561; A61B 5/055; G01R 33/50

(57) ABSTRACT

In a short T2 tissue imaging method and system, a magnetic resonance image is acquired that includes a short T2 tissue based on point-wise encoding time reduction with radial acquisition point-wise encoding time reduction with radial acquisition (PETRA) sequences, to obtain a first image, a T2 preparation pulse cluster is applied for suppressing a short T2 tissue signal between the PETRA sequences according to a predetermined interval of applying the T2 preparation pulse cluster, a magnetic resonance image is acquired that excludes the short T2 tissue based on the PETRA sequences applied with the T2 preparation pulse cluster, to obtain a second image; and a magnetic resonance image of the short T2 tissue is obtained based on the second image and the first image (e.g. by subtracting the second image from the first image).

### CROSS REFERENCE TO RELATED APPLICATIONS

This patent application claims priority to Chinese Patent Application No. 201911258002.X, filed Dec. 10, 2019, which is incorporated herein by reference in its entirety.

### BACKGROUND

### Field

The present disclosure relates to the field of magnetic resonance imaging, and in particular to a short T2 tissue imaging method, a short T2 tissue imaging system, and a magnetic resonance imaging system.

### Related Art

Magnetic resonance imaging is an imaging technique using a magnetic resonance phenomenon. The principle of magnetic resonance phenomenon mainly involves nuclei containing an odd number of protons, for example, hydrogen nuclei widely existing in a human body, the protons thereof being in a spin motion, like small magnets, and the small magnets having irregular axes of spin. If an external magnetic field is applied, the small magnets will be rearranged according to magnetic lines of force of the external magnetic field, and are specifically arranged in two directions, i.e. directions parallel to and anti-parallel to the magnetic lines of force of the external magnetic field. The direction parallel to the magnetic lines of force of the external magnetic field mentioned above is referred to as a positive longitudinal axis, and the direction anti-parallel to the magnetic lines of force of the external magnetic field mentioned above is referred to as a negative longitudinal axis. The nuclei only have a longitudinal magnetization component that has both a direction and an amplitude. Nuclei in the external magnetic field are excited by radio frequency (RF) pulses at a specific frequency such that the axes of spin of the nuclei deviate from the positive longitudinal axis or the negative longitudinal axis so as to produce resonance, which is the magnetic resonance phenomenon. After the axes of spin of the excited nuclei mentioned above deviate from the positive longitudinal axis or the negative longitudinal axis, the nuclei have a transverse magnetization component.

After the radio frequency pulses stop being transmitted, the excited nuclei transmit echo signals and gradually release the absorbed energy in the form of electromagnetic waves. Both the phase and energy level thereof are restored to the state before being excited, and the echo signals transmitted by the nuclei are subjected to further processing such as space encoding such that the image can be reconstructed. The above process of the excited nuclei being recovered to the state before being excited is referred to as a relaxation process, and the time required for recovery to an equilibrium state is referred to as a relaxation time.

The human body contains a variety of tissue elements. Imaging studies of short T2 tissues such as tendons, ligaments, and lungs are of great clinical and scientific significance. For these short T2 tissues, several magnetic resonance imaging (MRI) techniques have been proposed, including ultra-short echo time (UTE) imaging, point-wise encoding time reduction with radial acquisition (PETRA), and the like. In order to maximize the contrast and dynamic range of the short T2 tissues, effective suppression of long T2 tissues is also required. Although the single-echo PETRA technique can acquire an image of a short T2 tissue, it has limited suppression of the long T2 tissue.

With the development of magnetic resonance imaging technology, techniques such as dual-echo PETRA subtraction and dual inversion recovery ultra-short echo time (DIR UTE) have emerged. The dual-echo PETRA refers to: in order to obtain an image containing only signals from the short T2 tissues, another read-out gradient with an opposite polarity is applied at a second echo time TE2 to refocus the spin system to a second echo. In this way, one measurement produces two images, and subtraction of the two images can be performed to leave only the signals of the short T2 tissues. However, a total scanning time is about three times that of single-echo PETRA, so it is more sensitive to motion. DIR UTE refers to: two long adiabatic inversion pulses are used to suppress tissues with long T2. The first adiabatic inversion pulse reverses the magnetization intensity of long T2 water, and the second reverses the magnetization intensity of long T2 fat. Short T2 particles experience significant transverse relaxation during the long adiabatic inversion process, and are least affected by the inversion pulse.

In addition, those skilled in the art are still looking for other solutions.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 shows flowchart of a short T2 tissue imaging method according to an exemplary embodiment of the present disclosure.

FIG. 2 shows images obtained by imaging based on a PETRA sequence and a resulting magnetic resonance image according to an exemplary embodiment of the present disclosure.

FIG. 3 is a schematic diagram of a positional relationship between a T2 preparation pulse cluster and a PETRA sequence according to an exemplary embodiment of the present disclosure.

FIG. 4 is diagram of a short T2 tissue imaging system according to an exemplary embodiment of the present disclosure.

The exemplary embodiments of the present disclosure will be described with reference to the accompanying drawings. Elements, features and components that are identical, functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

An aspect of the present disclosure is to provide a short T2 tissue imaging method, and another aspect proposes a short T2 tissue imaging system and a magnetic resonance imaging system, to obtain a magnetic resonance image of a short T2 tissue.

A short T2 tissue imaging method proposed in an embodiment of the present disclosure comprises: acquiring a magnetic resonance image comprising a short T2 tissue based on point-wise encoding time reduction with radial acquisition PETRA sequences, to obtain a first image; applying a T2 preparation pulse cluster for suppressing a short T2 tissue signal between the PETRA sequences according to a predetermined interval of applying the T2 preparation pulse cluster, and acquiring a magnetic resonance image that does not comprise the short T2 tissue based on the PETRA sequences applied with the T2 preparation pulse cluster, to obtain a second image; and subtracting the second image from the first image to obtain a magnetic resonance image of the short T2 tissue.

In one implementation, the interval of applying the T2 preparation pulse cluster is determined according to longitudinal relaxation of the short T2 tissue between two adjacent T2 preparation pulse clusters and a total scanning time.

In one implementation, before said subtracting of the second image from the first image, the method further comprises: multiplying the second image by a predetermined scale factor to obtain a processed second image; and said subtracting of the second image from the first image comprises: subtracting the processed second image from the first image.

In one implementation, the scale factor is determined by using the following method: determining an empirical value as the scale factor; or dividing the first image by the second image to obtain a scale factor matrix; selecting, from the scale factor matrix, a plurality of candidate scale factors in an area of interest corresponding to a long T2 tissue, calculating an average of the plurality of candidate scale factors, and determining the average as the scale factor.

In one implementation, the T2 preparation pulse cluster includes: a first 90-degree hard pulse, an adiabatic pulse, and a second 90-degree hard pulse; wherein the first 90-degree hard pulse is applied along an X-axis to flip longitudinal magnetization along a Y-axis to a transverse plane; the adiabatic pulse is applied along the Y axis to refocus transverse magnetization flipped to the transverse plane; and the second 90-degree hard pulse is applied in the reverse direction along the X-axis to restore the refocused transverse magnetization to a Z-axis.

In one implementation, the method further comprises: after the second 90-degree hard pulse is applied, applying a spoiled gradient on the three axes X-axis, Y-axis, and Z-axis to remove the phase of residual transverse magnetization.

In one implementation, the method further comprises: after each PETRA sequence applied with the T2 preparation pulse cluster, applying a spoiled gradient on the three axes X-axis, Y-axis, and Z-axis to remove the phase of the residual transverse magnetization.

A short T2 tissue imaging system proposed in an embodiment of the present disclosure comprises: an image acquisition device, configured to acquire magnetic resonance image data comprising a short T2 tissue based on point-wise encoding time reduction with radial acquisition PETRA sequences; apply a T2 preparation pulse cluster for suppressing a short T2 tissue signal between the PETRA sequences according to a predetermined interval of applying the T2 preparation pulse cluster, and acquire magnetic resonance image data that does not comprise the short T2 tissue based on the PETRA sequences applied with the T2 preparation pulse cluster; and an image processing device, configured to perform image reconstruction on the magnetic resonance image data comprising the short T2 tissue to obtain a first image, perform image reconstruction on the magnetic resonance image data that does not comprise the T2 tissue to obtain a second image, and subtract the second image from the first image to obtain a magnetic resonance image of the short T2 tissue.

In one implementation, the interval of applying the T2 preparation pulse cluster is determined according to longitudinal relaxation of the short T2 tissue between two adjacent T2 preparation pulse clusters and a total scanning time.

In one implementation, before subtracting the second image from the first image, the image processing device further multiples the second image by a predetermined scale factor to obtain a processed second image; and subtracts the processed second image from the first image.

In one implementation, the scale factor is derived from an empirical value; or the image processing device divides the first image by the second image to obtain a scale factor matrix; selects, from the scale factor matrix, a plurality of candidate scale factors in an area of interest corresponding to a long T2 tissue, calculates an average of the plurality of candidate scale factors, and determines the average as the scale factor.

A magnetic resonance imaging system proposed in an embodiment of the present disclosure comprises the short T2 tissue imaging system described in any of the foregoing implementations.

It can be learned from the above solution that, because in the embodiments of the present disclosure, the T2 preparation pulse cluster is combined into single-echo PETRA, with appearance of the T2 preparation pulse cluster, the short T2 tissue appears dark due to the decay of T2, while the long T2 tissue has a limited signal drop. Therefore, a magnetic resonance image comprising the short T2 tissue can be first acquired based on the single-echo PETRA pulse sequence; the T2 preparation pulse cluster is then added between the PETRA pulses at a specific interval, to acquire a magnetic resonance image that does not comprise the short T2 tissue; and the magnetic resonance image comprising only the short T2 tissue can be obtained by subtracting the magnetic resonance image that does not comprise the short T2 tissue from the magnetic resonance image comprising the short T2 tissue. In this method, due to the short duration of the T2 preparation pulse cluster and the small time increment, the total scanning time of the two scans is approximately twice that of the original PETRA. This method has a higher time efficiency compared with the dual-echo PETRA method. In addition, since the two scans are carried out separately, if a motion occurs only in one scan, only the scan in which the motion occurs needs to be rescanned. Therefore, the time required for rescanning is relatively short, which is only 30% of the rescanning time of the dual-echo PETRA method. Moreover, this method inherits an advantage of the PETRA technique in being quiet, and is insensitive to b0 non-uniformity.

Further, by restoring the image of the long T2 tissue from the second image using a scale factor, a more prominent short T2 tissue image can be obtained.

Moreover, applying a spoiled gradient after and before the T2 preparation pulse cluster is applied can help to eliminate the phase of the residual transverse magnetization.

Finally, because quiet imaging is patient-friendly, it is a future imaging trend, and a PETRA sequence is by far the quietest sequence, but its application program is very limited. The technical solutions in the embodiments of the present disclosure can expand the application range of the PETRA sequence.

In an embodiment of the present disclosure, in order to achieve the effect of dual-echo PETRA, that is, to obtain an image containing only the short T2 tissue without consuming as much total scanning time as the dual-echo PETRA, it is considered to combine a T2 preparation pulse cluster into single-echo PETRA, because with appearance of the T2 preparation pulse cluster, the short T2 tissue appears dark due to the decay of T2, while the long T2 tissue has a limited signal drop. Therefore, a magnetic resonance image comprising the short T2 tissue can be first acquired based on the single-echo PETRA pulse sequence; the T2 preparation pulse cluster is then added between PETRA pulses at a specific interval to suppress the short T2 tissue, to acquire a magnetic resonance image that does not comprise the short T2 tissue; and the magnetic resonance image comprising only the short T2 tissue can be obtained by subtracting the magnetic resonance image that does not comprise the short T2 tissue from the magnetic resonance image comprising the short T2 tissue.

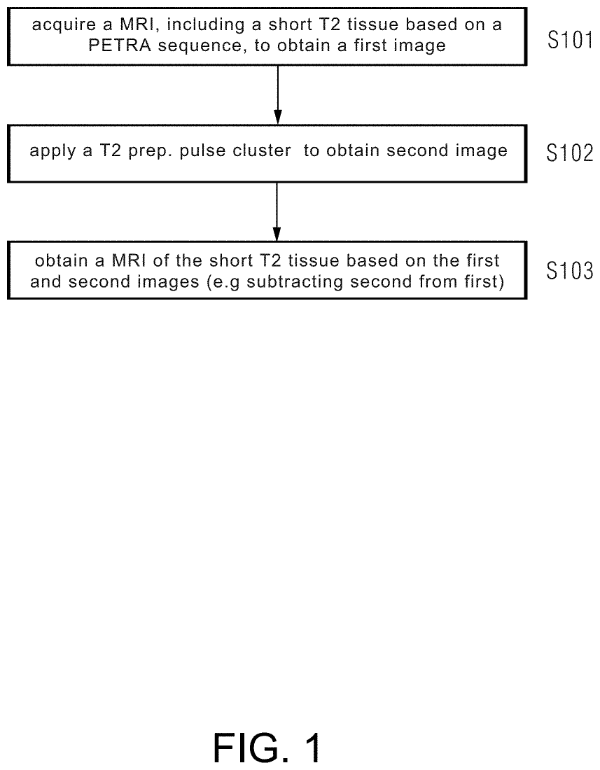

FIG. 1 is an exemplary flowchart of a short T2 tissue imaging method in an embodiment of the present disclosure. As shown in FIG. 1, the method may comprise the following steps.

Step S101: acquiring a magnetic resonance image comprising a short T2 tissue based on a PETRA sequence, to obtain a first image.

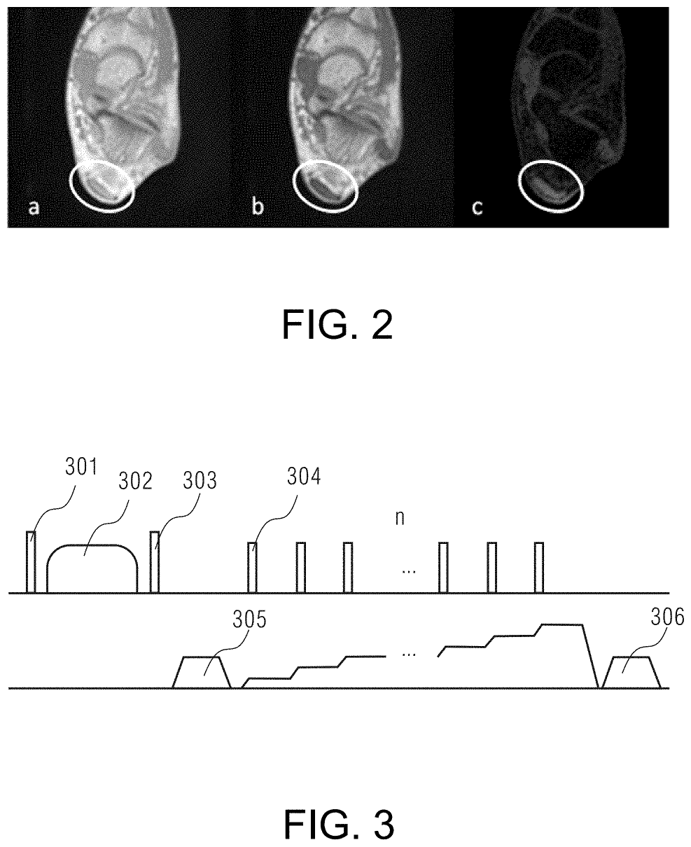

In this step, a single-echo PETRA sequence is used for magnetic resonance imaging as usual. As shown in an image part a on the left of FIG. 2, which shows a first image obtained by imaging based on the PETRA sequence on the 1.5 T Siemens Aera system with a 16-channel ankle coil in an example of the present disclosure, a short echo time of 0.07 ms is used, so that the short T2 tissue appears bright and can be well detected and displayed on the image. It can be seen that the tendon shown in the circle is bright.

Step S102: applying a T2 preparation pulse cluster for suppressing a short T2 tissue signal between the PETRA sequences according to a predetermined interval of applying the T2 preparation pulse cluster, and acquiring a magnetic resonance image that does not comprise the short T2 tissue based on the PETRA sequences applied with the T2 preparation pulse cluster, to obtain a second image.

In this step, the T2 preparation pulse cluster comprises: a first 90-degree hard pulse, an adiabatic pulse, and a second 90-degree hard pulse; wherein the first 90-degree hard pulse may be applied along an X-axis to flip longitudinal magnetization along a Y-axis to a transverse plane; the adiabatic pulse may be applied along the Y axis to refocus transverse magnetization flipped to the transverse plane; and the second 90-degree hard pulse may be applied in the reverse direction along the X-axis (or referred to as along the X-axis) to restore the refocused transverse magnetization to a Z-axis.

Each time after the T2 preparation pulse cluster is applied, the suppressed short T2 tissue signal will slowly recover. Therefore, the degree of recovery of the short T2 tissue signal, that is, the magnitude of the short T2 tissue signal, depends on a time elapsed since the T2 preparation pulse cluster is applied. However, applying more T2 preparation pulse clusters means more time consumption and a longer total scanning time, and therefore, it is necessary to balance the scanning time and image contrast. For this reason, the interval of applying the T2 preparation pulse cluster in the embodiment of the present disclosure is determined according to a longitudinal relaxation time (also called a longitudinal recovery time) T1 of the short T2 tissue and the total scanning time.

FIG. 3 is a schematic diagram of a positional relationship between a T2 preparation pulse cluster and a PETRA sequence in an embodiment of the present disclosure. As shown in FIG. 3, one T2 preparation pulse cluster is applied every n PETRA pulses 304. The one T2 preparation pulse cluster consists of a first 90-degree hard pulse 301, an adiabatic pulse 302, and a second 90-degree hard pulse 303. If the interval of applying the T2 preparation pulse cluster is represented by n*TR (wherein TR is a time between two pulses in the PETRA sequence, and may be called a repetition time; and n is the number of pulses), n*TR should not be too long, so that the longitudinal magnetization of the short T2 tissue signal will not recover too much; and n should not be too small either, otherwise it will significantly increase the total scanning time. In one embodiment, n may be set between 100 and 200.

In addition, in order to eliminate the phase of the residual transverse magnetization, as shown in FIG. 3, after each second 90-degree hard pulse is applied, a spoiled gradient 305 may be applied on the three axes X-axis, Y-axis, and Z-axis to remove the phase of residual transverse magnetization. Further, after n PETRA sequences are applied with the T2 preparation pulse cluster each time, another spoiled gradient 306 is applied on the three axes X-axis, Y-axis, and Z-axis, to further remove the phase of residual transverse magnetization.

As shown in an image part b in the middle of FIG. 2, which shows a second image obtained by imaging based on the PETRA sequence applied with a T2 preparation pulse cluster on the 1.5 T Siemens Aera system with a 16-channel ankle coil in an example of the present disclosure, the tendon shown in the circle is dark.

Step S103: subtracting the second image from the first image to obtain a magnetic resonance image of the short T2 tissue.

As shown in an image part c on the right of FIG. 2, which shows a magnetic resonance image obtained by subtracting the second image from the first image shown in FIG. 2, the tendon shown in the circle is highlighted.

In addition, considering that after the T2 preparation pulse cluster is applied, although the long T2 tissue has a limited signal drop, it is still a drop. In order to obtain a more prominent image of the short T2 tissue, the second image can be multiplied by a scale factor before step 103, to enhance the recovery of the image of the long T2 tissue in the second image. Then in step 103, the second image multiplied by the scale factor is subtracted from the first image.

The scale factor may be determined based on experience, for example, an empirical value may be determined as the scale factor. Alternatively, the scale factor may be determined by using the following method: dividing the first image by the second image to obtain a scale factor matrix; selecting, from the scale factor matrix, a plurality of candidate scale factors in an area of interest corresponding to a long T2 tissue, calculating an average of the plurality of candidate scale factors, and determining the average as the scale factor.

The short T2 tissue imaging method in the embodiments of the present disclosure has been described above in detail, and a short T2 tissue imaging system in the embodiments of the present disclosure will be described below in detail. The short T2 tissue imaging system in the embodiments of the present disclosure can be used to implement the short T2 tissue imaging method in the embodiments of the present disclosure. For details not disclosed in the system embodiment of the present disclosure, reference may be made to the corresponding description in the method embodiment of the present disclosure, and the details are not described herein again.

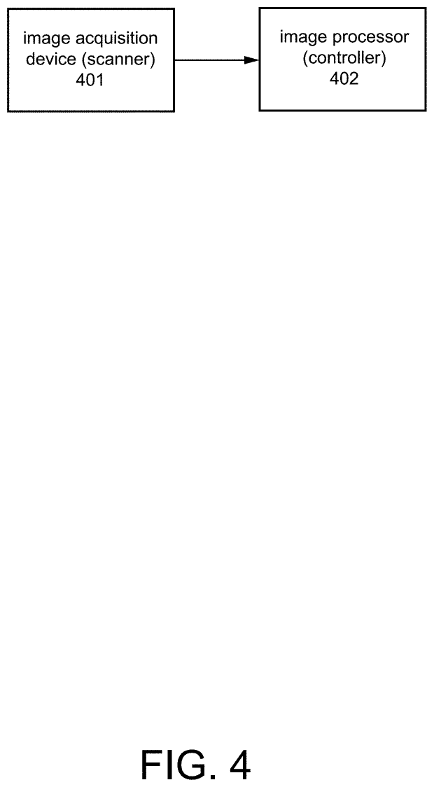

FIG. 4 is an exemplary structural diagram of a short T2 tissue imaging system in an embodiment of the present disclosure. As shown in FIG. 4, the short T2 tissue imaging system may comprise: an image acquisition device (scanner) 401 and an image processor (controller) 402.

The image acquisition device 401 is configured to acquire magnetic resonance image data comprising a short T2 tissue based on point-wise encoding time reduction with radial acquisition PETRA sequences; apply a T2 preparation pulse cluster for suppressing a short T2 tissue signal between the PETRA sequences according to a predetermined interval of applying the T2 preparation pulse cluster, and acquire magnetic resonance image data that does not comprise the short T2 tissue based on the PETRA sequences applied with the T2 preparation pulse cluster.

The image processing device/image processor 402, which may also be referred to as a controller, is configured to perform image reconstruction on the magnetic resonance image data comprising the short T2 tissue to obtain a first image, perform image reconstruction on the magnetic resonance image data that does not comprise the T2 tissue to obtain a second image, and subtract the second image from the first image to obtain a magnetic resonance image of the short T2 tissue. In an exemplary embodiment, the image processing device (controller) 402 includes processor circuitry that is configured to perform one or more functions and/or operations of the image processing device 402.

The interval of applying the T2 preparation pulse cluster may be determined according to longitudinal relaxation of the short T2 tissue between two adjacent T2 preparation pulse clusters and a total scanning time.

In one implementation, before subtracting the second image from the first image, the image processing device may further multiply the second image by a predetermined scale factor to obtain a processed second image; and subtract the processed second image from the first image.

The scale factor can be derived from an empirical value. Alternatively, the image processing device divides the first image by the second image to obtain a scale factor matrix; selects, from the scale factor matrix, a plurality of candidate scale factors in an area of interest corresponding to a long T2 tissue, calculates an average of the plurality of candidate scale factors, and determines the average as the scale factor.

In the embodiment of the present disclosure, a positional relationship between the T2 preparation pulse cluster and the PETRA sequence, and a position of applying a spoiled gradient may all be shown in FIG. 3, and the details are not described herein again.

A magnetic resonance imaging system proposed in the embodiments of the present disclosure may comprise the short T2 tissue imaging system described in any of the foregoing implementations.

According to aspects of the present disclosure, the T2 preparation pulse cluster is combined into single-echo PETRA, with appearance of the T2 preparation pulse cluster, the short T2 tissue advantageously appears dark due to the decay of T2, while the long T2 tissue has a limited signal drop. Therefore, a magnetic resonance image comprising the short T2 tissue can be first acquired based on the single-echo PETRA pulse sequence; the T2 preparation pulse cluster is then added between the PETRA pulses at a specific interval, to acquire a magnetic resonance image that does not comprise the short T2 tissue; and the magnetic resonance image comprising only the short T2 tissue can be obtained by subtracting the magnetic resonance image that does not comprise the short T2 tissue from the magnetic resonance image comprising the short T2 tissue. In this method, due to the short duration of the T2 preparation pulse cluster and the small time increment, the total scanning time of the two scans is approximately twice that of the original PETRA. This method advantageously has a higher time efficiency compared with the dual-echo PETRA method. In addition, since the two scans are carried out separately, if a motion occurs only in one scan, only the scan in which the motion occurs needs to be rescanned. Therefore, the time required for rescanning is relatively short, which is only 30% of the rescanning time of the dual-echo PETRA method. Moreover, this method inherits an advantage of the PETRA technique in being quiet, and is insensitive to b0 non-uniformity.

Further, by restoring the image of the long T2 tissue from the second image using a scale factor, a more prominent short T2 tissue image can be obtained.

Moreover, applying a spoiled gradient after and before the T2 preparation pulse cluster is applied can help to eliminate the phase of the residual transverse magnetization.

Finally, because quiet imaging is patient-friendly, it is a future imaging trend, and a PETRA sequence is by far the quietest sequence, but its application program is very limited. The technical solutions in the embodiments of the present disclosure can expand the application range of the PETRA sequence.

The above description is only the preferred embodiments of the present disclosure and is not intended to limit the present disclosure. Any modifications, equivalent substitutions, improvements, etc. made within the spirit and principles of the present disclosure shall fall within the scope of protection of the present disclosure.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

For the purposes of this discussion, the term “processor circuitry” shall be understood to be circuit(s), processor(s), logic, or a combination thereof. A circuit includes an analog circuit, a digital circuit, state machine logic, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

### REFERENCE LIST

S101-S103 operations
301 First 90-degree hard pulse
302 Adiabatic pulse
303 Second 90-degree hard pulse
304 PETRA pulse
305, 306 Spoiled gradient
401 Image acquisition device (scanner)
402 Image processing device (processor)

1. A short T2 tissue imaging method, comprising:
acquiring a magnetic resonance image including a short T2 tissue based on point-wise encoding time reduction with radial acquisition point-wise encoding time reduction with radial acquisition (PETRA) sequences, to obtain a first image;
applying a T2 preparation pulse cluster for suppressing a short T2 tissue signal between the PETRA sequences according to a predetermined interval of applying the T2 preparation pulse cluster, and acquiring a magnetic resonance image that excludes the short T2 tissue based on the PETRA sequences applied with the T2 preparation pulse cluster, to obtain a second image; and
obtaining a magnetic resonance image of the short T2 tissue based on the second image and the first image.

2. The short T2 tissue imaging method of claim 1, wherein obtaining the magnetic resonance image of the short T2 tissue comprises: subtracting the second image from the first image to obtain a magnetic resonance image of the short T2 tissue.

3. The short T2 tissue imaging method of claim 1, wherein the interval of applying the T2 preparation pulse cluster is determined according to longitudinal relaxation of the short T2 tissue between two adjacent T2 preparation pulse clusters and a total scanning time.

4. The short T2 tissue imaging method of claim 2, wherein:
before said subtracting of the second image from the first image, the method further comprises: multiplying the second image by a predetermined scale factor to obtain a processed second image; and
said subtracting of the second image from the first image comprises: subtracting the processed second image from the first image.

5. The short T2 tissue imaging method of claim 1, further comprising generating a processed second image based on the second image and a predetermined scale factor, wherein obtaining the magnetic resonance image of the short T2 tissue is based on processed second image and the first image.

6. The short T2 tissue imaging method of claim 1, wherein obtaining the magnetic resonance image of the short T2 tissue is further based on a predetermined scale factor.

7. The short T2 tissue imaging method of claim 4, wherein the predetermined scale factor is determined by:
dividing the first image by the second image to obtain a scale factor matrix;
selecting, from the scale factor matrix, a plurality of candidate scale factors in an area of interest corresponding to a long T2 tissue;
calculating an average of the plurality of candidate scale factors; and
determining the average as the scale factor.

8. The short T2 tissue imaging method of claim 4, further comprising determining an empirical value as the scale factor.

9. The short T2 tissue imaging method of claim 6, wherein the predetermined scale factor is determined by:
dividing the first image by the second image to obtain a scale factor matrix;
selecting, from the scale factor matrix, a plurality of candidate scale factors in an area of interest corresponding to a long T2 tissue;
calculating an average of the plurality of candidate scale factors; and
determining the average as the scale factor.

10. The short T2 tissue imaging method of claim 1, wherein the T2 preparation pulse cluster comprises: a first 90-degree hard pulse, an adiabatic pulse, and a second 90-degree hard pulse; wherein:
the first 90-degree hard pulse is applied along an X-axis to flip longitudinal magnetization along a Y-axis to a transverse plane;
the adiabatic pulse is applied along the Y-axis to refocus transverse magnetization flipped to the transverse plane; and
the second 90-degree hard pulse is applied in the reverse direction along the X-axis to restore the refocused transverse magnetization to a Z-axis.

11. The short T2 tissue imaging method of claim 10, further comprising: after the second 90-degree hard pulse is applied, applying a spoiled gradient on the three axes X-axis, Y-axis, and Z-axis to remove the phase of residual transverse magnetization.

12. The short T2 tissue imaging method of claim 11, further comprising: after the PETRA sequences are applied with the T2 preparation pulse cluster each time, applying a spoiled gradient on the three axes X-axis, Y-axis, and Z-axis to remove the phase of the residual transverse magnetization.

13. A computer program which includes a program and is directly loadable into a memory of an imaging device, when executed by a processor of the imaging device, causes the processor to perform the method as claimed in claim 1.

14. A non-transitory computer-readable storage medium with an executable program stored thereon, that when executed, instructs a processor to perform the method of claim 1.

15. A short T2 tissue imaging system, comprising:
a scanner configured to:
acquire magnetic resonance image data including a short T2 tissue based on point-wise encoding time reduction with radial acquisition (PETRA) sequences;
apply a T2 preparation pulse cluster for suppressing a short T2 tissue signal between the PETRA sequences according to a predetermined interval of applying the T2 preparation pulse cluster; and
acquire magnetic resonance image data that excludes the short T2 tissue based on the PETRA sequences applied with the T2 preparation pulse cluster; and
an image processor configured to:
perform image reconstruction on the magnetic resonance image data including the short T2 tissue to obtain a first image;
perform image reconstruction on the magnetic resonance image data that does not comprise the T2 tissue to obtain a second image; and
obtain a magnetic resonance image of the short T2 tissue based on the second image and the first image.

16. The short T2 tissue imaging system of claim 15, wherein the image processor is configured to subtract the second image from the first image to obtain the magnetic resonance image of the short T2 tissue.

17. The short T2 tissue imaging system of claim 15, wherein the interval of applying the T2 preparation pulse cluster is determined according to longitudinal relaxation of the short T2 tissue between two adjacent T2 preparation pulse clusters and a total scanning time.

18. The short T2 tissue imaging system of claim 16, wherein the image processor is further configured to:
multiple the second image by a predetermined scale factor to obtain a processed second image before subtracting the second image from the first image; and
subtract the processed second image from the first image to obtain the magnetic resonance image of the short T2 tissue.

19. The short T2 tissue imaging system of claim 18, wherein:
the scale factor is derived from an empirical value; or the image processor is configured to:
divide the first image by the second image to obtain a scale factor matrix;
select, from the scale factor matrix, a plurality of candidate scale factors in an area of interest corresponding to a long T2 tissue;
calculate an average of the plurality of candidate scale factors; and
determine the average as the scale factor.

20. A magnetic resonance imaging system, comprising the short T2 tissue imaging system of claim 15.
